(19) United States
(12) Patent Application Publication
(10) Pub. No.: US 2014/0194778 A1
(43) Pub. Date: Jul. 10, 2014

(54) SYRINGE ENABLED FOR ASPIRATING BLOOD INTO A SAMPLING SITE IN A CLOSED MANNER AND METHOD

(71) Applicant: BIOMETRIX LTD., Jerusalem (IL)

(72) Inventors: Elad UZIEL, Kadish Luz (IL); Yair MENDELS, Moza Elit (IL)

(73) Assignee: BIOMETRIX LTD., Jerusalem (IL)

(21) Appl. No.: 14/206,469

(22) Filed: Mar. 12, 2014

Related U.S. Application Data
(63) Continuation-in-part of application No. 13/296,327, filed on Nov. 15, 2011.
Application No. 13/296,327 is a continuation-in-part of application No. PCT/US2010/037043, filed on Jun. 2, 2010.
(60) Provisional application No. 61/183,886, filed on Jun. 3, 2009.

(51) Int. Cl. A61B 5/15

(52) U.S. Cl. CPC A61B 5/150992
USPC 600/578

(57) ABSTRACT

An in-line sampling syringe for selectively introducing and aspirating a fluid from a downstream fluid conduit connected to a catheterized patient. The syringe includes a housing with a distal component defining a distal chamber having a proximal opening and a distal outlet port to be coupled to a downstream fluid conduit; and a pair of proximal wall components assembled together in an airtight manner defining a proximal chamber having a proximal opening for coupling to an external source of fluid and a distal opening that is coupled to the proximal opening of the distal component. The syringe also includes an elongated volume regulator positioned inside the housing. A curved element is positioned at and in alignment with the distal outlet and retention elements are defined in the distal component at the distal outlet port to mount and retain the curved element at the distal outlet port while enabling flow around the curved element relative to the distal outlet port. A conduit extends from the proximal opening of the proximal chamber through the elongated volume regulator to the curved element at the distal outlet port.

### RELATED APPLICATIONS

This application is a continuation-in-part of U.S. patent application Ser. No. 13/296,327 filed Nov. 15, 2011, which is a continuation-in-part of PCT International Patent Application No. PCT/US2010/037043 filed Jun. 2, 2010, designating the United States of America and claiming priority from U.S. Provisional Patent Application No. 61/183,886 filed Jun. 3, 2009. This application also claims the benefit of U.S. Provisional Patent Application No. 61/414,427 filed Nov. 17, 2010, under 35 USC §119(e). The entire contents of each of the aforementioned applications are hereby incorporated by reference herein.

### FIELD OF THE INVENTION

The present invention relates to an In-Line sampling device and method for withdrawing (aspirating) blood from a patient fitted with a blood vessel catheter. More particularly, the present invention relates to a syringe enabled for aspirating blood into a sampling site in a closed manner, sealed from ambient air, in order to reduce the risk of human error and cross contaminations during the sampling process. Still more particularly, the present invention relates to a syringe, designed to operate manually or automatically and in synchronization with a sampling stopcock valve.

### BACKGROUND OF THE INVENTION

Sealed sterile blood sampling systems are known that have a dual function of introducing an upstream sterile fluid (e.g. saline solution) to a patient located downstream, and drawing back (aspirating) upstream fluid into the syringe. In a typical system, a small amount of infused fluid runs through the blood sampling line to the patient, when the line is not in use. This enables the blood sampling line to be maintained in a clear, unblocked/unclogged condition. When it is desired to take a blood sample from the patient, the fluid is aspirated beyond the sampling site so that a clean blood sample may be withdrawn. Relevant prior art documents that describe aspirating syringes or other syringe like aspirating devices said fluid sampling devices include the following.

U.S. Pat. No. 5,324,266 & U.S. Pat. No. 5,265,621 describe inline syringes which are operated linearly, namely the aspiration is performed by manually pulling the internal part of a syringe. This procedure may be physically strenuous due to the resistance of the gasket inside the syringe which may lead to accidental over aspiration. These patents further describe a sealing sleeve for maintaining a closed environment inside the syringe. This sleeve moves during aspiration, which can lead to ruptures or disengagement of the sleeve resulting in breach of the closed system. The linear operation of the syringe results in the extension of a piston beyond the syringe by a displacement equivalent to the aspirated volume, essentially doubling the length of the syringe. This is cumbersome in a hospital setting, and especially strenuous on a patient's arm. Additionally, operation of this “linear style” syringe requires the use of two hands, one to extract the piston, the other to prevent the syringe from moving.

U.S. Pat. No. 5,961,472 discloses a syringe which can be operated single handedly as opposed to the previous two patents discussed. However this syringe requires two independent squeezing motions instead of one, is not inline, and thus requires an additional valve system.

U.S. Pat. No. 5,374,401 discloses a blood sampling apparatus that uses a rotational movement to linear displacement of a piston via a threaded transmission system. This type of transmission involves high resistance due to friction of the thread. As a result high torque is exerted on a patient's arm (or stand) and could mask resistance of the fluid line due to occlusions. In addition this apparatus does not operate in-line and therefore requires an additional valve system, and is cumbersome to clean after aspiration. The exact amount of fluid aspirated is hard to determine as this apparatus does not incorporate a scale or any other measurement means.

U.S. Pat. No. 6,159,164 discloses a blood sampling system that does not operate in-line and therefore requires an additional valve system, and is cumbersome to flush and clean after aspiration. The exact amount of fluid aspirated is hard to determine as this apparatus does not incorporate a scale or any other measurement means. This system is operated by manually pushing an actuator towards the vertical axis of the apparatus which may result in torque that dislocates the apparatus from a patient's arm.

There are automatic systems for blood aspiration, for example, such as described in the U.S. Pat. No. 7,680,042 and US Published Patent Application No. US201010217154 and some of these systems incorporate use of integrated pressure monitoring. However, these systems cannot be operated manually, and most important, these systems do not operate in a closed manner, which allows the return of dead space fluids to patient, rather they discard these fluids as they are contaminated once aspired. In addition to the loss of fluids, working with systems that are not closed may result in higher contamination risks.

U.S. Pat. No. 5,758,643 is an example of a system that works in a closed manner and can return the dead space fluids to a patient; however, this system cannot operate manually and does not have an integrated pressure sensor for monitoring blood pressure.

None of the prior art described herein above enables the use of an automatic mode for aspirating syringes using an electronic system. Furthermore, pressure monitoring is required in many applications which require blood sampling; none of the prior art described above enable a device integrated pressure monitoring application.

### SUMMARY OF INVENTION

The configuration of the novel aspirating syringe of the present invention is a simpler construction that enables easier production and more efficient quality inspection. The force transferring parts of the novel syringe are physically separated from the fluid transfusion parts. Also, the construction provides an improvement in the ability of the novel syringe construction to convert a turning force into a linear force, which improves the ability of the novel syringe to draw the fluid and flush it out.

### BRIEF DESCRIPTION OF THE DRAWINGS

FIG. 1 shows schematically the assembled system of the present invention, wherein a syringe and sampling port device are coupled for in-line sampling and monitoring, and an auto pressurized cuff system retains infusion bag pressure;

FIG. 2a shows schematically an exploded perspective view of a syringe;

FIG. 2b shows schematically an assembled perspective view of the syringe of FIG. 2a;

FIG. 2c shows schematically a cross-sectional partial view of the syringe cut transversely along A-A of FIG. 2b, enlarged and in a perspective view

FIG. 3a shows schematically an assembled, partially cut perspective view of a syringe with the gasket at the distal end of the fluid chamber; and,

FIG. 3b shows schematically an assembled, partially cut perspective view of a syringe with the gasket shifted away from the distal end of the fluid chamber.

### DETAILED DESCRIPTION OF THE INVENTION

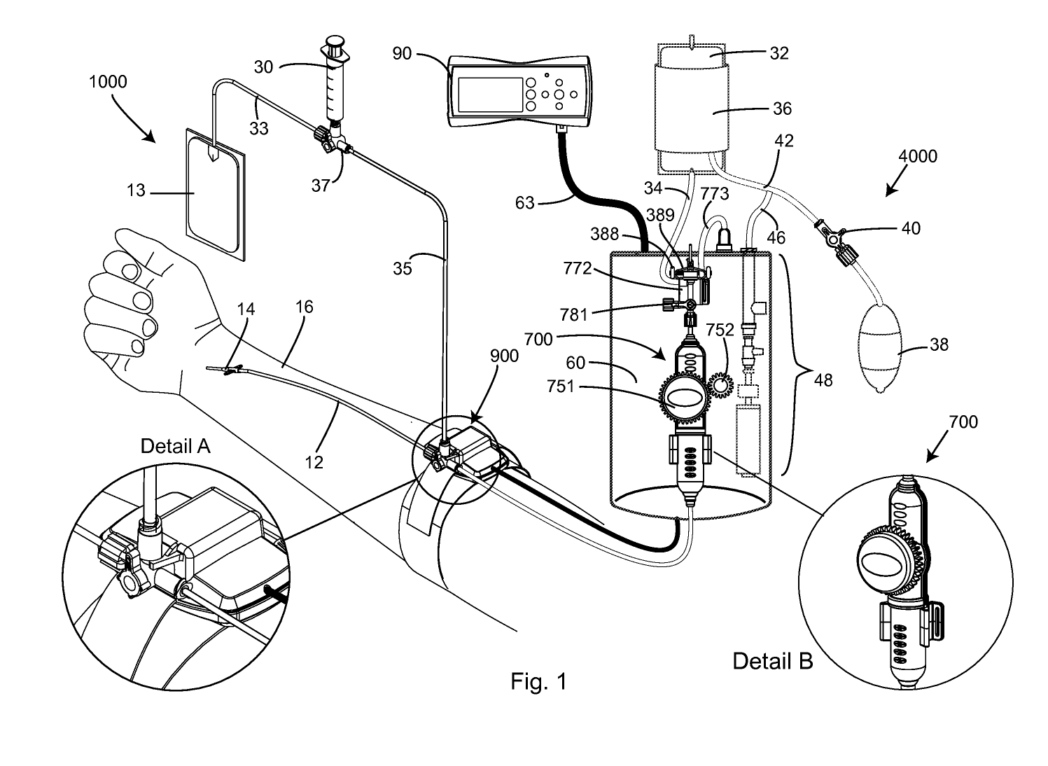

A preferred embodiment of the blood sampling system of the present invention is shown in FIG. 1, referred generally as (1000), and consists of an automatic blood pressure measuring system with a sampling mechanism. System (1000) consists of an IV bag (32) connected to a fluid line (12) leading from a catheter (14) embedded in a patient's arm (16). System (1000) comprises a sampling port mechanism (900) shown enlarged in Detail A, which is connected via side tube (35) to a stopcock valve (37). Stopcock valve (37) is connected to a sampling collecting syringe (30) or any other sample collecting vessel (not shown) and, connected through extension line (33), to a fluid waste collection bag (13) for in line analyzing of blood samples. A syringe (700) is mounted on an electronic apparatus (60). A cable (63) leads from electronic apparatus (60) to a remote controller and display (90). The remote controller (90) controls the synchronization between sample port (900) and syringe (700). A pressure management system (4000), comprising a pressure cuff (36) that surrounds IV bag (32) is inflated or pressure released via a manual pump (38) through valve (40) via line (42). A branch line (46) leads to an automatic pump (48) which is a part of electronic apparatus (60), both of which are described in detail in US 2012/0123298. A pressure transducer (772) external to syringe (700) is positioned upstream of syringe (700) and connects to IV bag (32) through extension line (34), and to a power supply via cable (773). Pressure transducer (772) comprises a side outlet (781) which is mechanically opened by a shaft (not shown), to enable the pressure of the inside of syringe (700) to be essentially equal to atmospheric pressure, for the purpose of calibration of pressure transducer (772). Mechanical finger (388) selectively opens and closes the flush lever (389) for selectively allowing and preventing fluid flow to syringe (700). Knob (751) is rotated manually as described herein below, or may be rotated by a wheel motor (752) as shown in FIG. 1, or via any other suitable mechanical or electronic means.

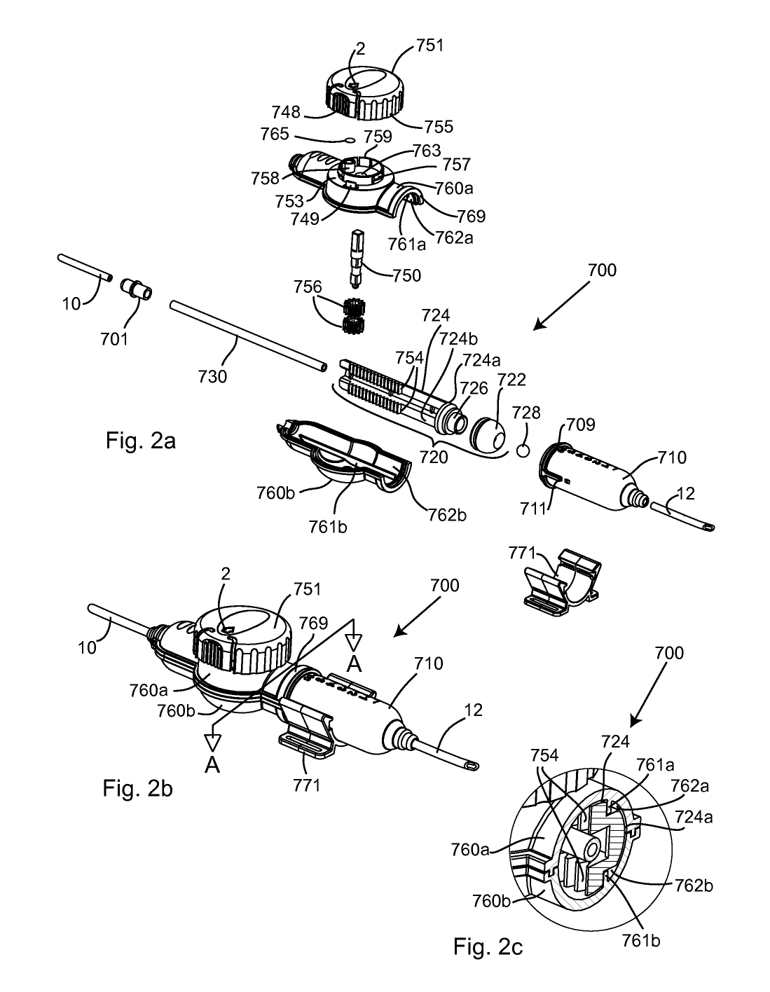

Syringe (700) is shown in an exploded view in FIG. 2a and in an assembled view in FIG. 2b. Syringe (700) includes a housing comprised of first and second housing walls (760a) (760b) and a fluid chamber (710), within which is a volume regulator (720), comprising a gasket (722) and a drive portion (724). The proximal end (709) of fluid chamber (710) is coupled with the distal end (769) of the joined housing walls (760a) (760b), as seen in FIG. 2b. Gasket (722) divides the internal space of fluid chamber (710) into a liquid side and an air side and prevents leakage of fluid out of the fluid side of fluid chamber (710). Gasket (722) is connected to drive portion (724) via coupling member (726) at the distal end of drive portion (724), as described herein below.

The shifting mechanism of drive portion (724) comprises a double rack (754) and pinion (756) actuator which translates the rotational motion of the knob (751) via shaft (750), into linear motion of drive portion (724) for shifting gasket (722) within fluid chamber (710). Alternatively, another suitable driving mechanism may be used for enabling the shifting of gasket (722).

FIG. 2c shows cross-sectional partial view of syringe (700) cut transversely along A-A of FIG. 2b, enlarged and in a perspective view. Referring particularly to FIG. 2c, the side (724a) of drive portion (724) opposite side (724b) of double rack (754), comprises a rounded contour corresponding to the contour of the inner surface (762a), (762b) of housing walls (760a), (760b), and a step portions cut longitudinally along the length of side (724a) of drive portion (724) to accommodate the off center ribs (761a), (761b) extending in a chord-wise direction from inner surface (762a), (762b) of each respective first and second housing wall (760a) (760b). Ribs (761a), (761b) and double pinions (756) maintain and guide drive portion (724) in position for riding along a fixed axial track when drive portion (724) shifts gasket (722) via the rack and pinions.

A fluid flow diverter (728) in the form of a ball is secured within fluid chamber (710) at its distal end, as described in greater detail herein below. Fluid flow diverter (728) creates a flow surface over which fluid may flow for washing away undesired substances, such as blood mixed with saline, from the distal end of fluid chamber (710).

A fluid tube (730) through which fluid is aspirated from and to a patient is disposed within syringe (700) along the central longitudinal axis and is connected to upstream fluid line (10) via adaptor (701), disposed within syringe (700). Downstream fluid line (12) extends out of the distal end of syringe (700).

A clip (771) for mounting syringe (700) on a mounting board (not shown) is removably attached to fluid chamber (710) via grooves (711) situated on opposing sides thereof.

Knob (751) is joined to syringe (700) at the central portion (753) of housing wall (760a). The underside of knob (751) has protrusions (not shown) that extend radially inward from the side wall (755), and when knob (751) is pressed over cylindrical extension (759) the knob protrusions snap over protrusions (757) that extend radially outward from the cylindrical extension (759). Projection (749) extending from central portion (753) of housing wall (760a) prevents knob (751) from rotating by blocking a corresponding projection (not shown) located at the inner surface of a segment (748) of the side wall (755) of knob (751). When segment (748) is pressed radially inward as indicated by arrow (2), knob (751) is unblocked and may be freely rotated about cylindrical extension (759) and thereby cause the rotation of pinion gears (756).

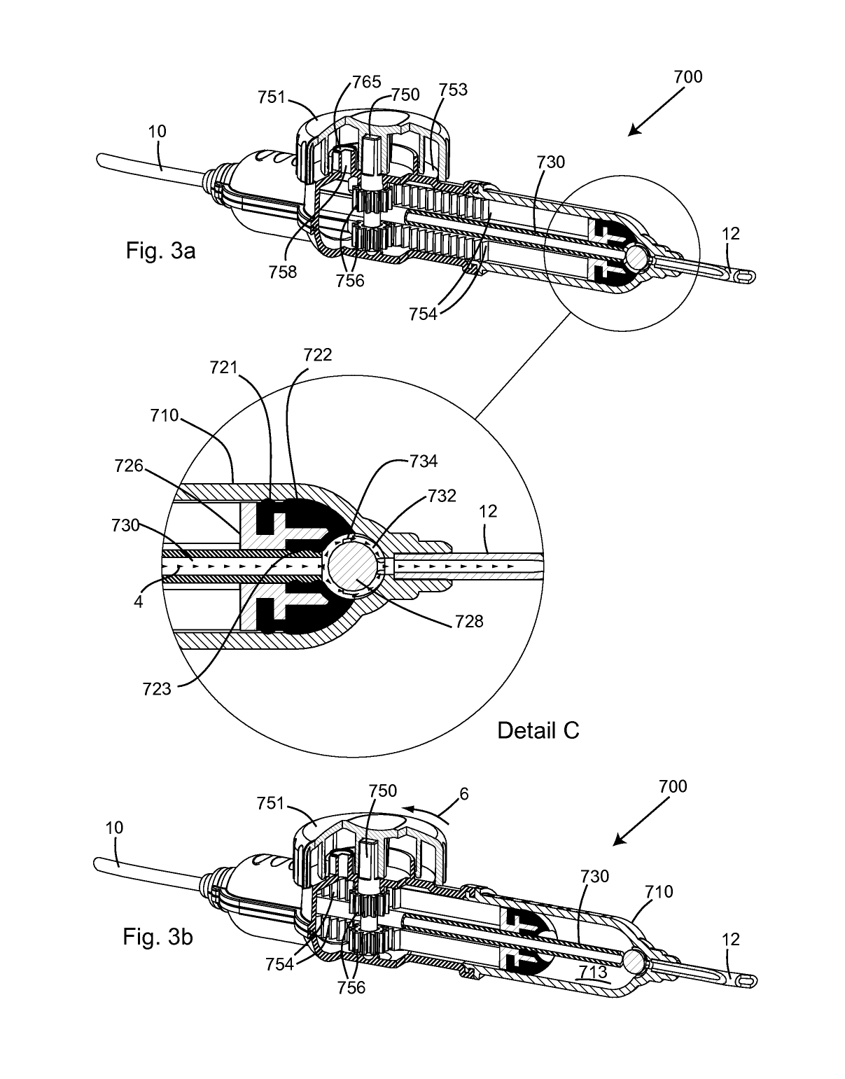

Central portion (753) of housing wall (760a) further comprises an opening (763) through which shaft (750) passes and is coupled with knob (751). This is best seen in FIG. 3a, which shows an assembled syringe (700), partially cut. Central portion (753) of housing wall (760a) also comprises a vent opening (758) for the purpose of admitting or discharging air from the air side of fluid chamber (710). A microbial (antibacterial) filter (765) prevents bacteria and/or other undesirable microorganisms from entering into the syringe housing when air is expelled out of filtered vent (765) during the aspiration process, as well as when air is sucked into the housing through filtered vent (765) when gasket (722) is shifted distally within fluid chamber (710). Filtered vent (765) or at least a portion thereof also is preferably treated with an anti-microbial agent, such as silver ions.

Referring to FIGS. 3a and 3b, the assembled syringe (700) is partially cut along fluid chamber (710) and housing walls (760a), (760b) to show two positions of gasket (722) within fluid chamber (710). In FIG. 3a gasket (722) is shown in contact with the distal end of the inner surface of fluid chamber (710) and in FIG. 3b gasket (722) is shown in a partially retracted position during fluid aspiration from a patient. The distal end of syringe (700) is shown enlarged in Detail C. As best seen in Detail C, fluid flow diverter (728) is secured within fluid chamber (710) at its distal end by ribs (732) which are integral with and protrude radially from the inner surface of fluid chamber (710). The radial curvature of ribs (732) is slightly less than the curvature of the fluid flow diverter (728). When pressing fluid flow diverter (728) between ribs (732), the tips (734) of ribs (732), which are separated from the surface of fluid chamber (710), are flexed outward so that fluid flow diverter (728) is securely disposed therebetween.

The cross sectional view of the distal end of syringe (700), best seen in Detail C, shows coupling member (726) at the distal end of drive portion (724) interlocked with gasket (722). Bulging portions (721), (723) of gasket (722) maintain a sealed relationship between gasket (722) and both the inner surface of fluid chamber (710) and the outer surface of fluid tube (730), respectively. Arrows (4) indicate fluid flow downstream through syringe (700), around fluid flow diverter (728) and out of downstream fluid line (12). Fluid passes over the inner surface of fluid chamber (710) to wash away undesired substances, including blood mixed with saline, as mentioned herein above.

With reference to FIG. 3b, when knob (751) is rotated in a counter clockwise direction, as indicated by arrow (6), volume regulator (720) is shifted proximally thereby causing aspiration of fluid into fluid side (713) of fluid chamber (710). As knob (751) is further rotated, the volume of fluid side (713) of fluid chamber (710) increases.

What is claimed is:

1. An in-line sampling syringe for selectively introducing a fluid to and aspirating a fluid from a downstream fluid conduit connected to a catheterized patient, said syringe comprising:
a. an elongated housing composed of (i.) a distal component defining a distal chamber having a proximal opening and a distal outlet port to be coupled to a downstream fluid conduit and (ii) a pair of proximal wall components assembled together in an airtight manner defining a proximal chamber having a proximal opening for coupling to an external source of fluid and a distal opening that is coupled to the proximal opening of the distal component;
b. an elongated volume regulator positioned longitudinally inside the housing that includes (i) a double rack having a distal extension with a gasket mounted on the distal end of said distal extension for separating a distal fluid chamber from a proximal air chamber, (ii) said elongated volume regulator being mounted for sliding longitudinally between a first position where the gasket is at the distal outlet port and a second position where the gasket is withdrawn from the distal outlet port, and (iii) a drive for the double rack;
c. a curved element positioned at and in alignment with the distal outlet;
d. retention elements defined in the distal component at the distal outlet port to mount and retain the curved element at the distal outlet port while enabling flow around the curved element relative to the distal outlet port; and
e. a conduit extending from the proximal opening of the proximal chamber through the elongated volume regulator to the curved element at the distal outlet port.

2. The syringe of claim 1 wherein the drive for the double rack comprises a pair of pinions supported on a shaft mounted on and between the pair of proximal wall components.

3. The syringe of claim 2 further including a turn wheel mounted on said shaft for shifting said gasket within said fluid chamber for adjusting the volume of said fluid chamber.

4. The syringe of claim 1 wherein an air relief element is mounted on one of the pair of proximal wall components.

5. The syringe of claim 4 wherein the air relief element has properties of one of antimicrobial and antibacterial.

6. The syringe of claim 1, wherein the conduit passes through said gasket.

7. The syringe of claim 3, wherein the turn wheel mounting on the shaft includes a detachable lock that can be unlocked singlehandedly to enable rotation of the turn wheel.

8. An in-line sampling system for selectively introducing a fluid to and aspirating a fluid from a downstream fluid conduit connected to a catheterized patient, said system comprising: a. a sampling valve having a first port for connecting to a downstream fluid conduit connected to a catheterized patient, a second port for sampling and a third port; b. a syringe according to claim 1 having its distal outlet port connected to the third port of the sampling valve, c. said fluid line being connectable to a flushing fluid supply via a valve mechanism; and d. a controller operatively selective for shifting the volume regulator to adjust the volume of said fluid chamber.

9. The in-line sampling system according to claim 8 further including a pressure measuring mechanism for monitoring pressure in said fluid line.

10. The in-line sampling system according to claim 8 further including an electronic controller operative for controlling monitoring of the fluid line, aspirating blood from a patient to the sampling valve and flushing the fluid line.

11. The in-line sampling system according to claim 10 wherein the electronic controller includes a motor coupled to the syringe, a stopcock sampling valve, a motor coupled to a stopcock and an electronic circuit for automatically synchronizing the stopcock sampling valve with the syringe and automatically stepping through a monitoring operation, an aspirating operation, a sampling operation and a flushing operation.

12. The in-line sampling system according to claim 8 wherein the mechanism for applying pressure to a flushing fluid supply includes a pressure cuff and a pump to apply pressure to the cuff and a valve to relieve pressure from the cuff.

13. The in-line sampling system according to claim 8 further including a pressure transducer to monitor the pressure of fluid at the sampling port of the sampling valve.

14. The syringe of claim 3 wherein the turn wheel includes a holding mechanism for detachably holding the turn wheel in a preselected position.

15. The syringe of claim 1 wherein the curved element is a ball mounted by the retention elements to enable fluid to flow around the ball.

16. The syringe according to claim 1 wherein the distal port is continuously open.

17. An in-line sampling system for selectively introducing a fluid to and aspirating a fluid from a downstream fluid conduit connected to a catheterized patient, said system comprising: a sampling valve having a first port for connecting to a downstream fluid conduit connected to a catheterized patient, a second port for sampling and a third port; an aspirating syringe according to claim 1 connected to the third port of the sampling valve, a pressure transducer for detecting an occlusion in the downstream fluid conduit created by a vacuum during aspiration which is caused by partial resistance increase or total occlusion in the downstream fluid conduit connected to a catheterized patient, and one of a display and audible alarm activated response to detection of an occlusion by the pressure transducer.

18. An in-line sampling system for selectively introducing fluid to and aspirating a fluid from a downstream fluid conduit connected to a catheterized patient, said system comprising: an aspirating syringe according to claim 1 shiftable between two positions, one for enabling a fluid to flow through and out the syringe and aspirating fluid into said syringe; a fluid reservoir for storing external fluid, and connected to said syringe via an upstream fluid conduit; a catheter for insertion into a patient, and connected to said syringe via a downstream fluid conduit; and, a sampling port situated along said downstream fluid conduit.
